(19) United States
(12) Patent Application Publication
(10) Pub. No.: US 2024/0335243 A1
(43) Pub. Date: Oct. 10, 2024

(54) CONTROL OF MOTION FOR MICRO-ROBOT USING COMMERCIAL GRADE MRI

(71) Applicant: BIONAUT LABS LTD., Herzliya (IL)

(72) Inventor: Michael SHPIGELMACHER, Los Angeles, CA (US)

(73) Assignee: BIONAUT LABS LTD., Herzliya (IL)

(21) Appl. No.: 18/681,480

(22) PCT Filed: Aug. 5, 2022
(86) PCT No.: PCT/US2022/039554

§ 371 Date: Feb. 5, 2024

Related U.S. Application Data
(60) Provisional application No. 63/231,145, filed on Aug. 9, 2021.

(51) Int. Cl. A61B 34/30; A61B 5/00; A61B 5/055; A61B 34/20; A61B 90/00; G01R 33/28

(52) U.S. Cl. CPC A61B 34/30; A61B 5/0042; A61B 5/055; G01R 33/285; A61B 2034/2051; A61B 2034/303; A61B 2090/3954

(57) ABSTRACT

The present disclosure provides methods of using a commercial grade magnetic resonance imaging (MRI) scanner to control and image motions of microbots in a subject. The method may further comprise a method of imaging to determine the location of the microbots in real time.

### FIELD OF THE INVENTION

The present disclosure is related in general to the field of magnetic navigation and imaging of micro-robots. In one embodiment, the present disclosure provides methods of using a commercial grade magnetic resonance imaging (MRI) scanner for magnetic navigation and imaging of micro-robots.

### BACKGROUND OF THE INVENTION

Although significant progress has been made in the development of cellular medicines and therapeutic drugs, challenges remain for their targeted delivery. While some therapeutics can be administered through localized delivery, systemic injection remains the best option for deep-seated targets or for multiple targets dispersed through the body. Shortcomings of systemic administration, however, include the challenge of localizing the therapeutic at the desired location, limited circulation time due to filtering of the blood by the lungs, spleen, liver and kidneys, as well as possible collateral damage when the therapeutic concentrates in an untargeted tissue.

To address these issues, navigation of millimeter-scale robots through the passageways of the body of a patient is being studied as a method to perform highly localized drug delivery or perform minimally invasive surgery. Untethered navigation can be achieved by placing a ferromagnetic piece inside the robot and producing a controlled magnetic field around a patient.

Propulsion and steering of millirobots can be accomplished by either moving a permanent magnet assembly around a patient or by controlling the current inside electromagnets. The latest solution is often realized with a magnetic resonance imaging (MRI) scanner which already includes several electromagnets. In an MRI, the background field magnetizes the ferrous components of the robot, and the gradient coils generate the magnetic gradient necessary to produce forces. The MRI scanner can be used simultaneously to provide real-time imaging of the operating area as well as positioning of the robot.

The size of magnetic microbots used in biological tissue (for example, liver, brain, eye, CSF, blood) ranges between 100s of microns to single cm. It is known that mm-scale micro-obots move efficiently in biological tissue (for example, brain tissue) with externally applied forces in the range of single mN or more. To generate such a magnetic force on the 100 micron-cm microbots, an external magnetic field gradient in the range of 100s of mT/m or more is needed. To control the motion of the microbot in a safe manner, the forces need to be controlled in three dimensions at a frequency of up to 20 Hz, allowing responses to temporal changes in the microbot location as imaged by a tracking modality. Available tracking modality for control of microbot motion in vivo include X-Ray (Fluoroscopy), Ultrasound, and other methods.

However, the nominal range of magnetic field gradients generated by commercial grade MRI is only up to <50 mT/m (or up to 200 mT/m in the most advanced investigational devices). This means that these machines do not generate the gradients required to drive the microbots efficiently in tissue (in the 100s of mT/m range). Moreover, most commercial MRI machines are not compatible with real time X-Ray or ultrasound imaging, making real time tracking of the microbot problematic. Other sensitive magnetic measurement methods would not be compatible with the MRI as it generates very strong, time varying magnetic fields. Lastly, introducing magnetic microbots on the 100 micron-single cm scale into an MRI machine is considered a safety hazard. In particular, the gradients in the MRI (either the BO gradient around the MRI, or the gradient fields generated by the gradient coils) represent a risk as these gradients could pull the microbot in an unsafe manner and harm tissue.

Currently, control of micro-robots in biological media relies on custom design of external hardware to generate the externally applied magnetic field. It would be desirable to leverage existing, clinically approved hardware for this purpose, reducing the need for custom hardware research and development, testing, regulatory approval, marketing, distribution and maintenance. Thus, in view of the challenges presented above, there is a need for improved methods of using commercial grade MRI machines to control microbots motion in an efficient and safe manner.

### SUMMARY OF THE INVENTION

Provided herein are methods of using a magnetic resonance imaging (MRI) scanner to

control motion of microbots in a subject, comprising the steps of: (a) introducing an MRI-safe lumen into a target anatomical area of the subject; (b) placing the subject with the MRI-safe lumen into the MRI scanner; (c) introducing microbot(s) through a high magnetic field gradient transition area to the subject through the MRI-safe lumen; and (d) operating the MRI scanner to control the motion of the microbot(s) in the subject.

Also provided herein are methods of using a magnetic resonance imaging (MRI) scanner to image microbots in a subject, comprises the steps of: (i) pre-scanning the subject in the MRI scanner to generate pre-scanned images; (ii) determining a location of the microbot(s) in real time; (iii) superimposing the pre-scanned image on the real time location of the microbot(s); and (iv) deducing a position of the microbots in reference to one or more MRI-visible fiducial markers in the subject's body.

These and other aspects of the invention will be appreciated from the ensuing descriptions of the figures and detailed description of the invention.

### DETAILED DESCRIPTION OF THE INVENTION

Untethered magnetic navigation of micro-robots within a human body using a magnetic resonance imaging (MRI) scanner is a promising technology for minimally invasive surgery or drug delivery. Depending on the intensity of the magnetic field, existing clinical MRI scanners are categorized into conventional (1-1.5 T), high-field (3-4 T) and ultra high-field (7-8 T). Higher field systems provide a higher signal to noise ratio and improve the imaging quality. However, as discussed above, there are challenges and hurdles to be overcome in using conventional MRI scanner for the control and imaging of microbots.

The present disclosure provides methods of using a commercial grade MRI scanner to control motion of microbots in a subject.

As used herein, the term “MRI-safe,” as in an “MRI-safe lumen” or an “MRI-safe adapter,” refers to a device which can be safely used in the vicinity or inside an operating MRI, in a way which is physically safe for a patient undergoing a medical procedure using the MRI and device in question. For example, an MRI safe lumen may be either made of non-magnetic materials (so it is not affected by an MRI), and/or physically fastened so it does not shift at all despite the operation of an MRI nearby.

In one aspect, the present disclosure provides a method of using a magnetic resonance imaging (MRI) scanner to control motion of microbots in a subject, comprising the steps of: (a) introducing an MRI-safe lumen into a target anatomical area of the subject; (b) placing the subject with the MRI-safe lumen into the MRI scanner; (c) introducing microbot(s) through a high magnetic field gradient transition area to the subject through the MRI-safe lumen; and (d) operating the MRI scanner to control the motion of the microbot(s) in the subject. In one embodiment, the subject is a human. In another embodiment, the subject is an animal. In one embodiment, the target anatomical area is liver, brain, or sub-arachnoid space.

In one embodiment, the MRI-safe lumen comprises a structure that prevents distortion of the lumen when it is subjected to a high gradient transition area of the MRI scanner. For example, the MRI-safe lumen comprises a flexible section inserted into the subject, and a rigid segment extending from inside the MRI scanner to a point outside the MRI scanner.

In one embodiment, the MRI-safe lumen further comprises an MRI-safe adaptor for mechanical introduction of microbots through the high gradient transition area into the target anatomical compartment. In one embodiment, the MRI-safe adaptor comprises a non-magnetic flexible grabber. In one embodiment of the above method, further comprising the step of guiding the microbot(s) back to the MRI-safe lumen and retrieving the microbot(s) with the mechanical adaptor. In one embodiment of the above method, the method further comprises the step of retracting the mechanical adaptor with the microbot(s) in a controllable fashion from the patient.

In one embodiment of the above method, the MRI gradient coils are operated at gradients of 500 mT/m-1,000 mT/m, maximal frequency of <10Hz, for maximal duration of <5 min, generating a force of single mN on a microbot of 1 cubic mm volume at a distance of 15 cm from the surface of the inner tube of the MRI.

In one embodiment, the above method further comprises a method of imaging the microbots, the imaging method comprises the steps of: (i) pre-scanning the subject in the MRI scanner to generate pre-scanned images; (ii) determining a location of the microbot(s) in real time; (iii) superimposing the pre-scanned image on the real time location of the microbot(s); and (iv) deducing a position of the microbots in reference to one or more MRI-visible fiducial markers in the subject's body, thereby determining a position for the microbot(s) in real time.

In another aspect, the present disclosure provides a method of using a magnetic resonance imaging (MRI) scanner to image microbots in a subject, comprises the steps of: (i) pre-scanning the subject in the MRI scanner to generate pre-scanned images; (ii) determining a location of the microbot(s) in real time; (iii) superimposing the pre-scanned image on the real time location of the microbot(s); and (iv) deducing a position of the microbots in reference to one or more MRI-visible fiducial markers in the subject's body. In one embodiment of the above method, step (iv) comprises triangulating the position of the microbots in reference to the fiducials in real time. In one embodiment of the above method, step (ii) comprises identifying a distortion in an MRI image due to an embedded magnetic component in the microbot(s). For example, the location of the microbot(s) is determined by calculating the geometrical center of the distortion.

The terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “a microbot” may include a plurality of microbots, including mixtures thereof.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. As used herein, the term “about” or “approximately” means within an acceptable error

range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value, such as an amount and the like, may encompass variations of ±20% or ±10%, more preferably ±5%, even more preferably ±1%, and still more preferably ±0.1% from the specified value, as such variations are appropriate to the disclosed values.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Control Motion of Microbots with Commercial Grade MRI

The nominal range of gradients generated by commercial grade MRI scanner is constrained

because of the need to switch the gradient rapidly (low KHz range) with a very high slew rate (50- 200 T/m/s) to generate the signal needed for accurate MRI soft tissue imaging. To achieve this very high slew rate and high frequency, high voltages and currents are fed into the gradient coils from the gradient amplifiers (in the range of up to 2500 V and up to 1000 Amp). Typically, an MRI scan takes 15-45 minutes. Running such a high current for such a long period, at high frequencies, results in significant heating of the coils (including eddy current effects). As gradient coils are typically water cooled, the maximal gradient is limited to avoid overheating, while still being high enough to generate the MRI images.

In contrast, the microbot control disclosed herein does not require the same set of parameters. In fact, available data describes control of microbots in the frequency range of 10 Hz or less (corresponding to the frame rate of available in vivo imaging modalities), at which eddy current effects are minimal, and operation for a period of up to 2 min at maximum current at a time. A required lower slew rate of up to 20 T/m/s (lower than the slew rate of the MRI) is assumed. The reduction of frequency by a factor of 500-1000 and the reduction of operating time by a factor of 7-20 results in a reduction of effective coil resistance by a factor of 2-5 and a reduction of heat energy by a factor of 25-100 over the course of the procedure, for a given current. This, in turn, allows a sustained maximal voltage for a longer period in every duty cycle, reaching effective currents which are 5-10 times higher than the maximal operating current of the MRI today, using the same hardware or slightly modified hardware (e.g., to support currents that are 5-10 times higher). Given that current is linear in gradient, this would translate to gradients of >5 times the maximal gradient of the given MRI machine, i.e., in the range of multiple 100s of mT/m. It should be noted that given the reduction of resistance by a factor of 2-5, a current can be achieved which is 2-5 times higher with the same feeder voltage from the gradient amplifier, maximizing system efficiency. At the slew rate of the existing system, if the time at maximal voltage is increased by a factor of 10, a maximal representative gradient of 1 T/m would be reached within <20 milliseconds, a short time frame relevant to frequency of 10 Hz (matching a cycle of 100 milliseconds), making this a practical design for gradient based control of microbots.

It has also been reported that metallic components embedded in tissue are seen in MRI images as distortion of the image (for example, large dark spots). This is typically viewed as an impediment to the use of the microbots inside an MRI. However, for the purpose of controlling the microbots, real-time soft tissue imaging is not required. It is possible to calculate the geometrical center of the distortion in the MRI image (or any other image processing technique) in real time to deduce the location of the microbots in real time. In one embodiment, it is possible to pre-scan the patient in the MRI and then superimpose the pre-scanned image on the real time location of the microbots, utilizing MRI-visible fiducial markers on the patient body, triangulating the position of the microbots in reference to the fiducials in real time. It should be noted the microbots with an embedded magnetic component in the range of 100s of micron or more generate a clearly visible distortion in the image, making this a practical method.

The range of BO gradients in the operating region of the MRI is in the single microT/m range, making this field very stable and reducing any risk for uncontrolled microbot motion.

However, the remaining risk is the transition from the outside of the MRI into the operating region inside the MRI. In this transition region, BO has gradients as high as 5T/m. In one aspect, this problem can be addressed and overcome in a method comprising the following steps:
a) The patient is pre-retreated with a standard interventional technique, introducing an MRI-safe lumen (for example, a sheath or a catheter) into a target anatomical area (for example, sub arachnoid space, liver, brain), prior to inserting the patient into an MRI.
b) The patient is inserted into the MRI where the MRI-safe lumen is accessible safely from outside the MRI, and has a structure preventing distortion of the lumen when subjected to internal forces of up to 1 N in the high gradient transition area. For instance, the lumen may have a flexible section inserted into the patient, and a rigid segment extending from inside the MRI to a safe distance away from the MRI, fastened to a fixture inside the MRI and outside the MRI.
c) The MRI-safe lumen is equipped with an MRI-safe adaptor configured to mechanically introduce the microbot(s) through the high gradient transition area and through the lumen into the target anatomical compartment in the body of the patient in a controllable fashion.
d) Once the microbot is in the right location in the patient body, it is released. Note that in that area the BO gradient is low so there is no risk of uncontrolled motion.
e) Upon completion of the procedure the microbot is guided back to the insertion spot and grabbed by the mechanical adaptor.
f) The mechanical adaptor is retracted in a controllable fashion from the patient and the MRI, mirroring the insertion method.

In one embodiment, the mechanical adaptor may be a non-magnetic flexible grabber. In one embodiment, the grabber is pre-loaded with a microbot, introduced into the lumen and advanced into the MRI through the high gradient transition area. The lumen does not allow the microbot to move laterally, while the grabber prevents the microbot from moving towards the MRI.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of controlling the motion of one or more magnetic microbots in a subject, the method comprising the steps of:
(a) providing a magnetic resonance imaging (MRI) scanner, the MRI scanner comprising a main magnet, one or more shim coils, a gradient system, a RF system, and a controller configured to direct operation of the other components of the MRI system, the MRI system further comprising a tube defining a hollow bore for receipt therein of the subject during use;
(b) introducing an MRI-safe lumen leading toward a target anatomical area of the subject at a distal end thereof;
(c) introducing the one or more microbots into the lumen at a proximal end thereof; and
(d) operating the MRI scanner to produce a varying magnetic gradient on the lumen, thereby controlling the motion of the one or more microbots through the lumen.

2. The method of claim 1, wherein the MRI scanner is operated at a frequency of no more than 20 Hz.

3. The method of any one of the preceding claims, wherein the MRI scanner is operated at a slew rate of no more than about 40 T/m/s.

4. The method of any one of the preceding claims, wherein the MRI scanner is operated for no more than 5 minutes to control the one or more microbots to traverse the lumen between it two ends.

5. The method of any one of the preceding claims, wherein the MRI scanner generates a force of between about 200 mN and about 1 N on the microbot at a distance of less than about 20 cm from a subject-facing surface of the tube MRI scanner.

6. The method of any one of the preceding claims, wherein each of the one or more microbots has a volume not exceeding about 1 mm³.

7. The method of any one of the preceding claims, wherein the operating of the MRI scanner further comprising an imaging process, the imaging process comprising:
(i) obtaining a base image of the subject;
(ii) determining, using the MRI scanner, real-time locations of the one or more microbots; and
(iii) superimposing microbot images, each corresponding to the determined real-time location of one of the microbots.

8. The method of claim 7, wherein obtaining the base image comprises pre-scanning the subject using the MRI scanner.

9. The method of any one of claims 7 and 8, the base image comprising a plurality of fiducial locations, the imaging process further comprising providing MRI-visible fiducial markers at positions corresponding to the fiducial locations in the base image, wherein determining the real-time locations of the one or more microbots comprises determining the locations of the fiducial markers and correlating them with the fiducial locations in the base image.

10. The method of claim 9, wherein determining the real-time locations of the one or more microbots comprises triangulating the position of each of the one or more microbots with reference to the fiducials in real time.

11. The method of any one of claims 7 through 10, wherein wherein determining the real-time locations of the one or more microbots comprises identifying a distortion in an MRI image due to an embedded magnetic component in each of the microbot(s).

12. The method of claim 11, wherein the location of each of the microbots is determined by calculating the geometrical center of the distortion.

13. The method of any one of the preceding claims, wherein the MRI-safe lumen comprises a structure that prevents distortion of the lumen when it is subjected to the magnetic field gradient arising at a transition area of the MRI scanner.

14. The method of any one of the preceding claims, wherein the MRI-safe lumen comprises a flexible section inserted into the subject, and a rigid segment extending from inside the MRI scanner to a point outside the MRI scanner.

15. The method of any one of the preceding claims, wherein the MRI-safe lumen further comprises an MRI-safe adaptor for mechanical introduction of microbots through the high magnetic field gradient transition area into the target anatomical compartment.

16. The method of claim 15, wherein the MRI-safe adaptor comprises a non-magnetic flexible grabber.

17. The method of any one of claims 15 and 16, further comprising guiding the microbot(s) back to the MRI-safe lumen and retrieving the microbot(s) with the mechanical adaptor.

18. The method of any one of claims 15 through 17, further comprising retracting the mechanical adaptor with the microbot(s) in a controllable fashion from the patient.

19. The method of any one of the preceding claims, wherein the subject is a human. 20 The method of claim 19, wherein the target anatomical area is in the brain.

21. The method of claim 20, wherein the target anatomical area is the subarachnoid space. 22 The method of claim 19, wherein the target anatomical area is selected from a group consisting of the liver, eye, ear, neck, lungs, pancreas, kidney, nasal cavity, mouth, GI tract, bladder, and stomach.

23. A method of imaging a microbot within a subject using an MRI scanner, the method comprising:
(i) obtaining a base image of the subject;
(ii) determining, using the MRI scanner, real-time locations of the one or more microbots;
(iii) superimposing microbot images, each corresponding to the determined real-time location of one of the microbots; and
(iv) operating the MRI scanner to produce a varying magnetic gradient on the lumen, thereby controlling the motion of the one or more microbots through the lumen.

24. The method of claim 23, wherein obtaining the base image comprises pre-scanning the subject using the MRI scanner.

25. The method of any one of claims 23 and 24, the base image comprising a plurality of fiducial locations, the imaging process further comprising providing MRI-visible fiducial markers at positions corresponding to the fiducial locations in the base image, wherein determining the real-time locations of the one or more microbots comprises determining the locations of the fiducial markers and correlating them with the fiducial locations in the base image.

26. The method of claim 25, wherein determining the real-time locations of the one or more microbots comprises triangulating the position of each of the one or more microbots with reference to the fiducials in real time.

27. The method of any one of claims 23 through 26, wherein wherein determining the real- time locations of the one or more microbots comprises identifying a distortion in an MRI image due to an embedded magnetic component in each of the microbot(s).

28. The method of any one of claims 23 through 27, wherein wherein determining the real- time locations of the one or more microbots comprises identifying a distortion in an MRI image due to an embedded magnetic component in each of the microbot(s).

29. The method of any one of claims 23 through 28, wherein the MRI scanner is operated at a frequency of no more than 20 Hz.

30. The method of any one of claims 23 through 29, wherein the MRI scanner is operated at a slew rate of no more than about 40 T/m/s.

31. The method of any one of claims 23 through 30, wherein the MRI scanner is operated for no more than 5 minutes to control the one or more microbots to traverse the lumen between it two ends.

32. The method of any one of claims 23 through 31, wherein the MRI scanner generates a force of between about 200 mN and about 1 N on the microbot at a distance of less than about 20 cm from a subject-facing surface of the tube MRI scanner. 33 The method of any one of claims 23 through 27, wherein each of the one or more microbots has a volume not exceeding about 1 mm³.
